(19) United States
(12) Patent Application Publication
(10) Pub. No.: US 2018/0235999 A1
(43) Pub. Date: Aug. 23, 2018

(54) PHYSICAL MULTI-IMPACT LIQUID-STATE ENERGY WATER, MANUFACTURING METHOD THEREOF AND DEVICE APPLYING THE SAME

(71) Applicant: KLEAN ENERGY CORPORATION LIMITED, Taichung City (TW)

(72) Inventors: Li-Tu WANG, Taichung (TW); Yu-Tang SHU, Taipei City (TW)

(21) Appl. No.: 15/436,007

(22) Filed: Feb. 17, 2017

(51) Int. Cl. A61K 33/00; C02F 9/00; A61K 41/00; A61F 9/00; A61M 35/00; A44C 5/00; G02C 11/00

(52) U.S. Cl. CPC A61K 33/00; C02F 9/00; A61K 41/0004; C02F 1/001; A61M 35/00; A44C 5/0023; G02C 11/00; A61F 9/0026

(57) ABSTRACT

A manufacturing method of a physical multi-impact liquid-state energy water is provided. The manufacturing method includes processing water by a coarse filtering process, a first standing process, a fine filtering process, a second standing process, a centrifugation process, a third standing process and a spiral pressurization process. The physical multi-impact liquid-state energy water is manufactured by repeated multiple impacts, and is water in small molecular groups having micro-vibration element liquid-state energy and having a value of frequency of nuclear magnetic resonance in full width at half maximum under 100 Hz. Water molecular groups of nearby regions are activated by physical resonance to miniaturize the water molecular groups of the nearby regions, in a way that the size of the water molecular groups of the nearby regions is fundamentally changed. The present invention is applicable to various products to promote maintaining body health.

### FIELD OF THE INVENTION

The present invention relates to water molecular groups, and more particularly to water in small molecular groups, manufacturing method thereof, and a device applying the water in small molecular group.

### BACKGROUND OF THE INVENTION

Water is an indispensible in the daily life of humans, and quality water is an essential factor for maintaining good health. Sources of water include tap water, well water and river water, whose multiple water molecules gather and form various types of water molecular groups through hydrogen bonding. Water molecular groups from different water sources differ in size. Researches and studies have shown that drinking water from water sources having small water molecular groups is beneficial to health due to following reasons. Dispersibility of small water molecular groups is high, such that drinking water in small water molecular groups promotes dispersion of red blood cells. Further, permeability of small water molecular groups is high, and so water in small water molecular groups is capable of easily passing through cell walls to nourish cells to further reduce the viscosity of the human blood. Moreover, being capable of enhancing biochemical effects in the human body and boosting activities of various enzymes, water in small water molecular groups may decompose blood lipids and reduce the blood liquid index, hence promoting health.

A detection method of employing the frequency of nuclear magnetic resonance in full width at half maximum is commonly and globally used to test the size of water molecular groups. In general, the size of a water molecular group gets smaller as the value of frequency of nuclear magnetic resonance in full width at half maximum is smaller, and gets larger as the value of frequency of nuclear magnetic resonance in half width at full maximum is larger. The value of frequency of nuclear magnetic resonance in full width at half maximum of tap water, well water, river water and rainwater is usually above 100 Hz or even as high as 157 Hz. Generally speaking, a larger frequency value of nuclear magnetic resonance in full width at half maximum means that the water contains a larger amount of pollutants or non-ideal ions such as sodium. As a result, the number of clusters formed in the water molecular groups is also larger; that is, the size of water molecular groups is larger, and such water is considered having poor water quality.

### SUMMARY OF THE INVENTION

It is an object of the present invention to disclose physical multi-impact liquid-state energy water, a manufacturing method thereof, and a device manufactured by incorporating the physical multi-impact liquid-state energy water. The present invention is capable of activating water molecular groups of nearby regions by physical resonance to miniaturize the water molecular groups of the nearby regions, in a way that the size of the water molecular groups is fundamentally changed. Thus, the present invention is applicable to various types of products to help maintaining body health.

To achieve the above object, the present invention provides physical multi-impact liquid-state energy water, which is manufactured by processing water by a coarse filtering process, a first standing process, a fine filtering process, a second standing process, a centrifugation process, a third standing process and at least one spiral pressurization process. The physical multi-impact liquid-state energy water, made by repeated multiple impacts, is in small water molecular groups having micro-vibration element liquid-state energy, and a value of frequency of nuclear magnetic resonance in full width at half maximum that is below 100 Hz. Further, the present invention is capable of activating water molecular groups of nearby regions using physical resonance to miniaturize the water molecular groups of the nearby regions, thereby fundamentally changing the size of the water molecular groups of the nearby regions.

The water may be selected from tap water, well water and river water. The coarse filtering process is having the water pass a fiber filter cartridge, preferably a fiber filter cartridge having a 5 μm aperture. The first standing process is allowing the water stand for at least six hours. The fine filtering process is having the water further pass an activated carbon filter cartridge and a KDF filter cartridge. The second standing process is allowing the water to stand for at least six hours. The centrifugation process is placing the water into a rotating bucket that rotates at a speed of 1800 to 6000 rpm per minute to cause the water to rotate and display a swirling distribution, and the water is drawn from a top portion of the swirling distribution of the water to complete the centrifugation process. The third standing process is allowing the water to stand for at least six hours. The spiral pressurization process is causing the water to enter a spiral pressure bucket at a speed of 3.2 to 5.2 meter per second (m/s). The spiral pressure bucket includes a tube, and an entrance and an exit respectively located at two ends of the tube. The center of the tube is provided with a column, which is surrounded by a spiral guide plate. The water enters the tube from the entrance, impacts the spiral guide plate, repeatedly impacts in a gap between the spiral guide plate and the tube, and flows out from the exit through the guiding of the spiral guide plate, hence completing the manufacture of the physical multi-impact liquid-state energy water.

The present invention may be applied to various products, such as portable worn products (eye masks and bracelets), water processing devices, and air fresheners. By filling a container by the physical multi-impact liquid-state energy water, a main body structure of the various products may be completed. Meanwhile, appropriate changes are made based on application needs of the various products. The container may be made from different materials, such as plastic bags, metals, plastic housing and ceramic materials.

Taking an eye mask for example, the eye mask includes a glass frame and two container housings. The glass frame includes a frame rim and two temples respectively connected to two sides of the frame rim. The two container housings are filled by the physical multi-impact liquid-state energy water and installed on the glass frame. Taking a bracelet for example, the bracelet includes a hollow loop, which is filled by the physical multi-impact liquid-state energy water. Taking a water processing device or an air freshener for example, the water processing device or air freshener includes a hollow housing, which is filled by the physical multi-impact liquid-state energy water.

Accordingly, with the above process, the physical multi-impact liquid-state energy water may be manufactured from common water, and then be accordingly made into portable worn products (e.g., eye masks and bracelets), water processing devices and air fresheners. These products do not need electric power and contains no chemical additives, and thus feature functions of energy-saving, carbon reduction and environmental protection and safety. The physical multi-impact liquid-state energy water activates water molecular groups of nearby regions by physical resonance, such that water in larger water molecular groups of the nearby regions is miniaturized into water in smaller water molecular groups to increase dissolved oxygen in the water. Further, water processed by the present invention preserves the freshness of vegetables and fruits, suppresses all anaerobic bacteria, changes the pH value of the processed water from slightly acidic to slightly alkaline (pH 7 to 8.5), and promotes the blood circulation of the human body and increases the blood flow by more than 20%. Moreover, the present invention eliminates odors and increases dissolved oxygen to freshen air. Therefore, the present invention satisfies different needs for promoting the human body health.

### BRIEF DESCRIPTION OF THE DRAWINGS

FIG. 1 is a flowchart of the present invention;

FIG. 2 is a schematic diagram of a rotating bucket of the present invention;

FIG. 3 is a schematic diagram of a spiral pressure bucket of the present invention;

FIG. 4 is a curve diagram of the frequency of a nuclear magnetic resonance in full width at half maximum of molecular groups of the present invention;

FIG. 5 is a schematic diagram of a structure according to a first embodiment of the present invention;

FIG. 6 is a schematic diagram of a structure according to a second embodiment of the present invention; and

FIG. 7 is a schematic diagram of a structure according to a third embodiment of the present invention.

### DETAILED DESCRIPTION OF THE PREFERRED EMBODIMENTS

Structural designs and technical details of the present invention are given with the accompanying drawings below.

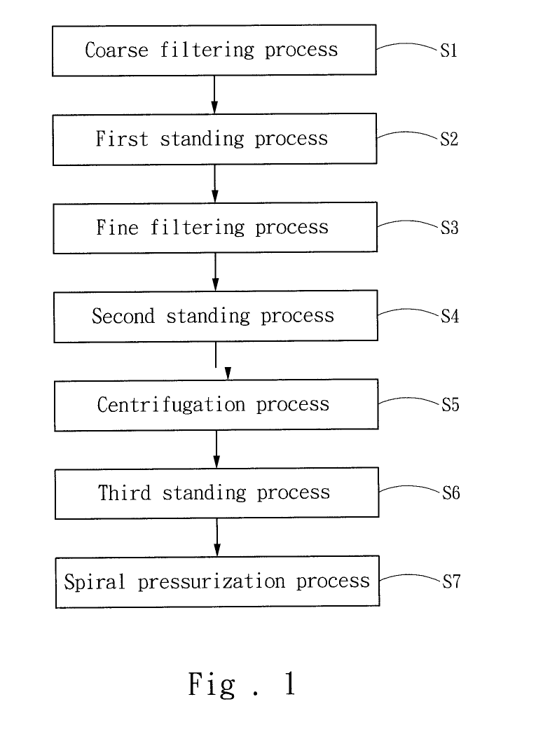

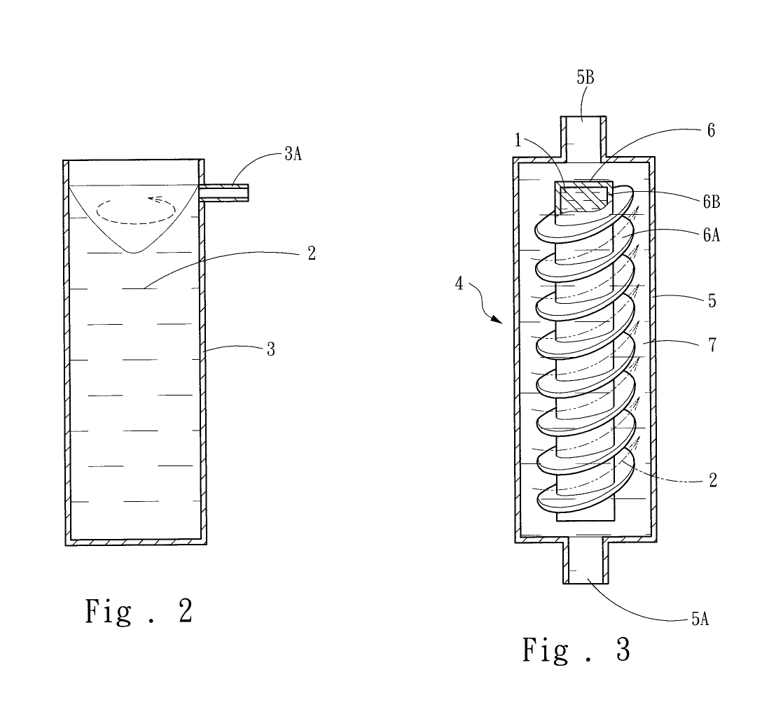

Referring to FIG. 1, FIG. 2 and FIG. 3, a manufacturing method of physical multi-impact liquid-state energy water 1 of the present invention includes processing water 2 by steps of: a coarse filtering process S1, a first standing process S2, a fine filtering process S3, a second standing process S4, a centrifugation process S5, a third standing process S6 and at least one spiral pressurization process S7, to manufacture the physical multi-impact liquid-state energy water 1.

The water 2 is selected from tap water, well water and river water, and may be any water source used in the daily life of humans. The coarse filtering process S1 is having the water 2 pass a fiber filter cartridge, preferably a fiber filtering cartridge having a 5 μm aperture, and serves a main purpose of filtering out impurities such as iron rust, silt, sand, hair, organic matters, algae and suspended microorganisms. The first standing process S2 is allowing the water 2 to stand for at least six hours, and serves a main purpose of standing the water 2 to achieve homogenization of the water 2. The fine filtering process S3 is having the water 2 further pass an activated carbon filter cartridge and a Kinetic Degradation Fluxion (KDF) filter cartridge. The activated carbon filter cartridge effectively absorbs chemical substances such as chlorine, pesticides, trihalomethane, odors, magnesium and organic substances. The KDF filter cartridge, through electron exchange oxidation reduction, removes heavy metals and organic pollutants from the water and suppresses bacteria growth, and may be implemented by an activated carbon composite filter cartridge to reinforce the absorption function. The KDF filter cartridge further provides functions of absorbing harmful substances including colorants and fluorides. The second standing process S4 is similarly allowing the water 2 to stand for at least six hours to achieve homogenization of the water 2.

The centrifugation process S5 is placing the water 2 into a rotating bucket 3. The rotating bucket 3 rotates at a speed of 1800 to 6000 rpm per minute to have the water 2 display a spiral distribution. The water 2 is drawn from a top portion of the spiral distribution of the water 2 to complete the centrifugation process S5. A drawing pipe 3A may be disposed on the rotating bucket 3 to draw the water 2. The third standing process S6 is allowing the water 2 to stand for at least six hours, to similarly achieve homogenization of the water 2.

The spiral pressurization process S7 has the water 2 enter a spiral pressure bucket 4 at a flowing speed of 3.2 m to 5.2 meters per second (m/s). The spiral pressure bucket 4 includes a tube 5, and an entrance 5A and an exit 5B respectively located at two ends of the tube 5. The center of the tube 5 is provided with a column 6, which is surrounded by a spiral guide plate 6A. The water 2 enters the tube 5 from the entrance 5A, impacts the spiral guide plate 6A, repeatedly impacts in a gap 7 between the spiral guide plate 6A and the tube 5, and flows out from the exit 5B through the guiding of the spiral guide plate 6A. Because the column 6 is freely placed into the tube 5, the column 6 also vibrates as the water 2 impacts the spiral guide plate 6A in a way that the impact frequency of the water 2 is increased. Further, to increase the number of impacts of the water 2, the spiral pressurization process S7 may use multiple spiral pressure buckets 4 for impacts and to prolong the impact time and hence increasing the number of times of the impacts. In general, three spiral pressure buckets 4 are provided in a preferred embodiment. Further, a standing process performed on the water 2 may be added between different spiral pressure buckets 4, so as to achieve homogenization of the water 2 using the standing process.

Further, to accelerate the process of manufacturing the physical multi-impact liquid-state energy water 1 from the water 2, the column 6 may include an internal space 6B. The physical multi-impact liquid-state energy water 1 may be placed in the internal space 6B to exercise the physical resonance effect on the physical multi-impact liquid-state energy water 1, thereby accelerating the process of manufacturing the physical multi-impact liquid-state energy water 1 from the water 2.

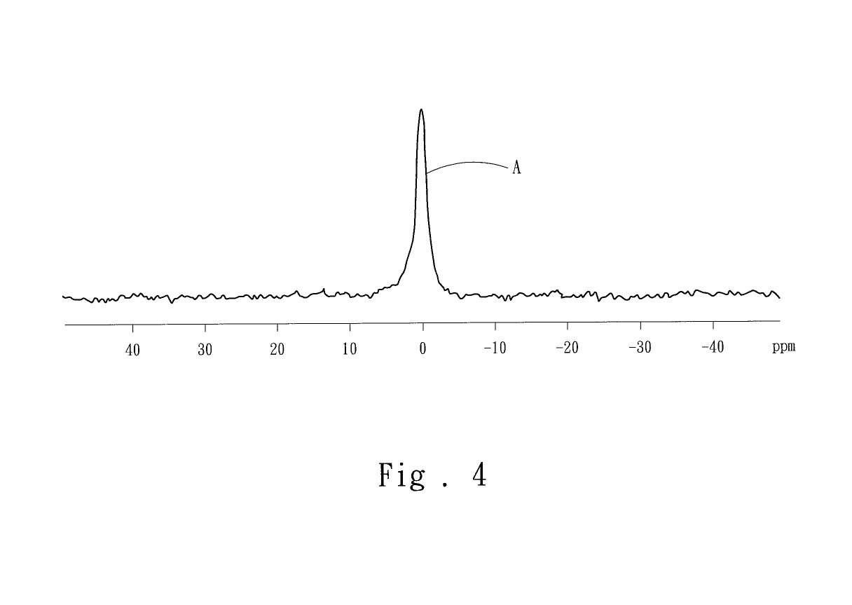

As previously described, the physical multi-impact liquid-state energy water 1 is manufactured by repeated multiple impacts, includes small water molecular groups having micro-vibration element liquid-state energy, and has a value of frequency of nuclear magnetic resonance in full width at half maximum kept below 100 Hz. Thus, using physical resonance, water molecular groups of nearby regions may be activated to miniaturize the water molecular groups of the nearby regions. Based on experimental data of Hungkuang University, the frequency of the nuclear magnetic resonance in full width at half maximum of the physical multi-impact liquid-state energy water 1 of the present invention, as measured by a nuclear magnetic resonance spectrometer, is 72.3±5 Hz. A principle involved is that, using the magnetism of atomic nuclei in the elements, when placed in a strong magnetic field, one single energy gap is split into a larger number of quantum states, and appropriate high-frequency magnetic waves are absorbed, while low quantum states are excited into high quantum states—such phenomenon is referred to as nuclear magnetic resonance. Further, regarding parameters used in this experiment, the pulse program is z30, the number of times of experiment scanning is 256 times, the acquisition time is 0.16389 second (s), the experiment temperature is 289.5 K, the experimental nuclear species is 17O, the relaxation time is 0.30000001 s, the pulse width is 10.00 μs, the spectrometer frequency of O1 and the spectrometer frequency are respectively 54.246373 Hz and 54.2463392 Hz, the window function is exponential multiplication (EM), and the Lorentz broadening is 20 Hz. Under the above experiment conditions, the frequency of 17O in full width at half maximum is detected, and the frequency in full width at half maximum that the 17O of the internal standard material absorbs is 20 Hz, which needs to be subtracted from all data detected to obtain actual data. The actual data is recorded and compared to obtain a curve A as shown in FIG. 4, which shows that the frequency of a nuclear magnetic resonance in full width at half maximum is 72.3±5 Hz.

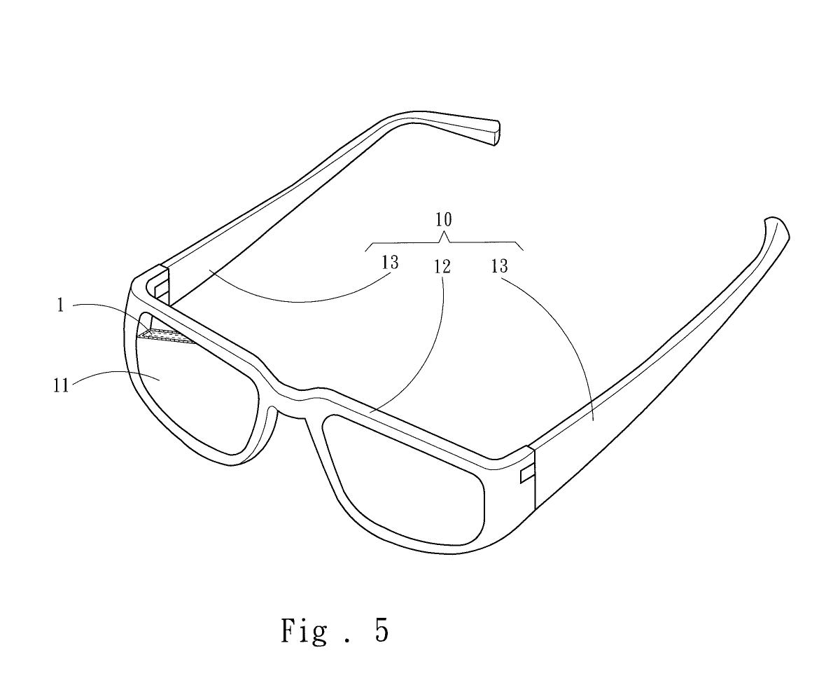

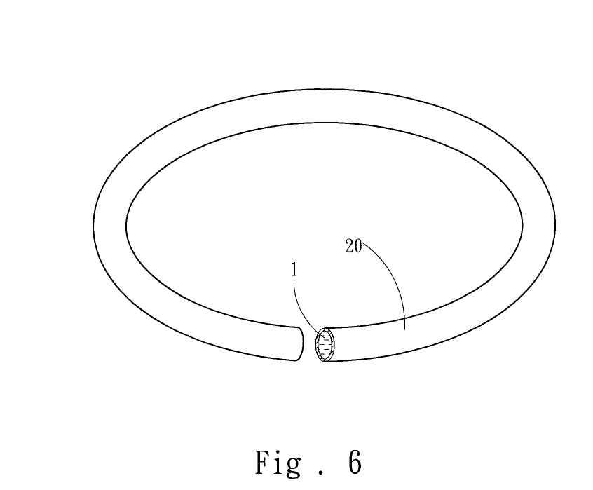

Some examples of applications of the physical multi-impact liquid-state energy water 1 of the present invention are given below. Referring to FIG. 5 and FIG. 6 showing a first embodiment and a second embodiment of the present invention, the present invention may be applied to manufacture portable worn products, such as an eye mask (the first embodiment) and a bracelet (the second embodiment). As shown in FIG. 5, a glasses-type eye mask includes a glass frame 10 and two container housings 11. The glass frame 10 includes a frame rim 12 and two temples 13 respectively connected to two sides of the frame rim 12. The two container housings 11 are filled by the physical multi-impact liquid-state energy water 1, and are installed on the frame rim 12. Thus, when worn by a user, the physical multi-impact liquid-state energy water 1 is caused to be close to eye sockets of the user. As the eye sockets of the human body are densely covered by tiny blood vessels, the physical multi-impact liquid-state energy water 1 may activate the blood vessels at the eye sockets by physical resonance to efficiently promote blood circulation of the human body and increase the blood flow by more than 20%, thereby effectively improving dark eye circles and small wrinkles as well as quickly eliminating eye fatigue.

Referring to FIG. 6, a bracelet includes a hollowing loop 20, which is filled by the physical multi-impact liquid-state energy water 1. Accordingly, when the bracelet is worn by a user, the physical multi-impact liquid-state energy water 1 is brought close to a wrist of the user. Because the main artery passes the wrist of the user, the physical multi-impact liquid-state energy water 1 may activate the artery at the wrist by physical resonance to efficiently promote blood circulation of the human body and increase the blood flow by more than 20%, thereby effectively improving the issue of blood lipid accumulation.

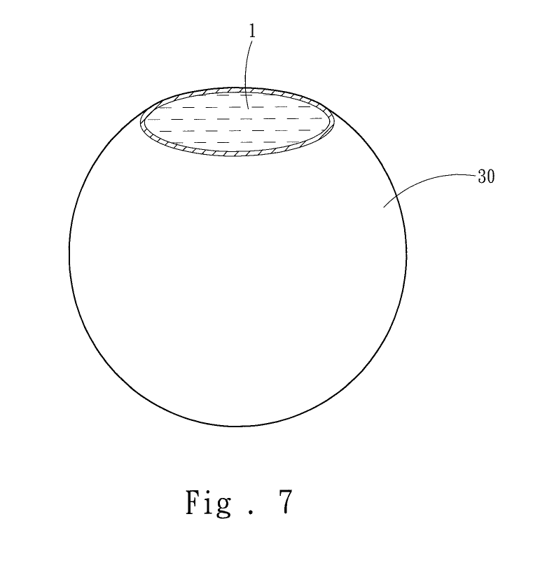

Referring to FIG. 7, a hollow housing 30 shown may be an egg-shaped body, a spherical body, a column-shaped body or a block-shaped body, and may be made of a material selected from the group consisting of a plastic bag, a metal, a plastic housing and a ceramic material. In FIG. 7, a spherical body is shown as an example. The hollow housing 30 is filled by the physical multi-impact liquid state energy water 1. Accordingly, the hollow housing 30 may be used as a water processing device when stored water (not shown) is placed therein, and the physical multi-impact liquid-state energy water 1 may activate the stored water by physical resonance to miniaturize the water molecular groups of the stored water and increase the dissolved oxygen in the water. Further, the processed water provides a preservation function for keeping the freshness of vegetables and fruits (prolonging the preservation time by more than 20%). Meanwhile, the processed water is also capable of suppressing anaerobic bacteria, and changes the pH value of the water from slightly acidic to slightly alkaline (pH 7 to 8.5). Based on test reports of water samples provided by Environmental Science Research and Study Center of Yuan Ze University, it is known from results of the report number B1050985-1 (order number YZ1051B0985) that, the stored water having been processed by the present invention has a concentration index (pH value) of hydrogen ions of 7.7 and a dissolved oxygen concentration of 7.5 mg/L, whereas the water quality of common tap water has a concentration index of hydrogen ions of 6.5 to 7 and a dissolved oxygen concentration of 4 to 5 mg/L. It is apparent that the present invention significantly increases the pH value and the dissolved oxygen.

Further, the hollow housing 30 may serve as an air refresher when placed in a closed space (e.g., a room or a refrigerator). Water molecular groups in the air may be activated by the physical multi-impact liquid-state energy water 1 by physical resonance, so that the water molecular groups may generate physical resonance with the water molecular groups in the air. That is, without involving electric power or chemicals, the water molecular groups in the air may be brought to the surface of the hollow housing 30 by the flowing of the wind. After the water molecular groups in the air are activated and miniaturized by impacts of the physical resonance, dissolved oxygen is increased to freshen the air. Meanwhile, hazardous odors and gases in the air are quickly decomposed to purify the air to provide people with fresh air to breathe.

In conclusion, the physical multi-impact liquid-state energy water of the present invention includes at least following advantages.

1. The present invention features functions of energy-saving, carbon reduction and environmental protection and safety without requiring electric power or chemical additives. 2. The present invention increases dissolve oxygen in water. 3. The water processed by the present invention preserves the freshness of vegetables and fruits (by prolonging the preservation time by more than 20%). 4. The water processed by the present invention suppresses all anaerobic bacteria. 5. The present invention changes the pH value of water from slightly acidic to slightly alkaline (pH 7 to 8.5). 6. The present invention converts water in large molecular water groups to water in small water molecular groups. 7. The water processed by the present invention promotes the blood circulation of the human body and increases the blood flow by more than 20%.

What is claimed is:

1. A manufacturing method of physical multi-impact liquid-state energy water, for manufacturing the physical multi-impact liquid-state energy water from water, the method comprising:
a coarse filtering process of having the water pass a fiber filter cartridge;
a first standing process of allowing the water to stand for at least six hours;
a fine filtering process of having the water to further pass an activated carbon filter cartridge and a KDF filter cartridge;
a second standing process of allow the water to stand for at least six hours;
a centrifugation process of placing the water into a rotating bucket, the rotating bucket rotating at a speed of 1800 to 6000 rpm per minute to have the water rotate and display a spiral distribution, and drawing the water from a top portion of the spiral distribution of the water to complete the centrifugation process;
a third standing process of allowing the water to stand for at least six hours; and
a spiral pressurization process of having the water enter a spiral pressure bucket at a flowing speed of 3.2 m to 5.2 meters per second (m/s), the spiral pressure bucket comprising a tube and an entrance and an exit respectively located at two ends of the tube, a center of the tube being provided with a column that is surrounded by a spiral guide plate; the water entering the tube from the entrance, impacting the spiral guide plate, repeatedly impacting in a gap between the spiral guide plate and the tube, and flowing out from the exit through guiding of the spiral guide plate, hence completing manufacturing the physical multi-impact liquid-state energy water.

2. The manufacturing method of claim 1, wherein the spiral pressurization process uses a plurality of the spiral pressure buckets for impacts, and a standing process on the water is added between different spiral pressure buckets.

3. The manufacturing method of claim 1, wherein the column comprises an internal space, which is placed with the physical multi-impact liquid-state energy water.

4. A physical multi-impact liquid-state energy water manufactured from the manufacturing method of claim 1, the physical multi-impact liquid-state energy water being water in small molecular groups having micro-vibration element liquid-state energy and having a value of frequency of nuclear magnetic resonance in full width at half maximum under 100 Hz.

5. A device manufactured by incorporating the physical multi-impact liquid-state energy water of claim 1, comprising:
a container, filled by the physical multi-impact liquid-state energy water.

6. A device manufactured by incorporating the physical multi-impact liquid-state energy water of claim 1, comprising:
a glass frame, comprising a frame rim and two temples respectively connected to two sides of the frame rim; and
two container housings, filled by the physical multi-impact liquid-state energy water and installed on the frame rim.

7. A device manufactured by incorporating the physical multi-impact liquid-state energy water of claim 1, comprising:
a hollow loop, filled by the physical multi-impact liquid-state energy water.

8. A device manufactured by incorporating the physical multi-impact liquid-state energy water of claim 1, comprising:
a hollow housing, filled by the physical multi-impact liquid-state energy water.

9. The device according to claim 8, wherein the hollow housing is in any shape selected from an egg shape, a spherical shape, a column shape and a block shape.

10. The device according to claim 8, wherein the hollow housing is in a material selected from the group consisting of a plastic bag, a metal, a plastic housing and a ceramic material.
